(19) United States
(12) Patent Application Publication
(10) Pub. No.: US 2003/0166234 A1
(43) Pub. Date: Sep. 4, 2003

(54) A METHOD FOR PREPARING MASSIVELY RmlC PROTEIN AND PURIFIED RmlC PROTEIN OF MYCOBACTERIUM TUBERCULOSIS BY IT

(76) Inventor: Tae-Yoon Lee, Taegu-shi (KR)

Correspondence Address: MERCHANT & GOULD PC, P.O. BOX 2903, MINNEAPOLIS, MN, 55402-0903, US

(21) Appl. No.: 10/088,728

(22) PCT Filed: Jul. 23, 2001
(86) PCT No.: PCT/KR01/01251

(30) Foreign Application Priority Data
Jul. 22, 2000 (KR) 2000/42274
Nov. 8, 2000 (KR) 2000/66236

(51) Int. Cl. C12N 9/24; C07H 21/04; C12N 15/74; C12P 21/02; C12N 1/21

(52) U.S. Cl. 435/200; 435/69.1; 435/252.33; 536/23.2; 435/320.1

(57) ABSTRACT

This invention relates to a mass preparation method of RmlC protein and the purified RmlC protein by the said method. RmlC is a gene product of rmlC, which is one of the biosynthesis genes of rhamnose that is an important element that consists cell wall of Mycobacterium tuberculosis. To be more specific, this invention improves the following disadvantage of recombinant RmlC protein of Mycobacterium tuberculosis previously reported that contains unnecessary 15 amino acids into the natural RmlC protein. This invention relates to a recombinant plasmid, a recombinant E. coli that is transformed by the plasmid, preparation method of Mycobacterium tuberculosis RmlC recombinant protein using the recombinant E. coli, purification method of recombinant RmlC protein, and the RmlC protein purified by the said method.

### TECHNICAL FIELD

This invention relates to a mass preparation method of RmlC protein, a gene product of rmlC that is one of the genes synthesizing rhamnose, which is an important element that constitutes cell wall of Mycobacterium tuberculosis. To be more specific, this invention relates to an improvement of disadvantages such as expression efficiency reduction of RmlC protein for the development of anti-tuberculosis drug(s) because of the fusion of 15 unnecessary amino acids into Mycobacterium tuberculosis RmlC recombinant protein as reported previously (Stern R. J. et al. Microbiology 145:663-671(1999)), problems of crystal formation because of the fusion of unnecessary amino acids when crystal of RmlC protein is formed, and extension of required time because of the fusion of unnecessary amino acids when the structure of RmlC protein is determined by X-ray crystallography. This method contains a recombinant plasmid that expresses RmlC protein itself without unnecessary amino acids and a method that can produce RmlC protein of Mycobacterium tuberculosis in large quantities that contains enzymatic activity of the corresponding RmlC protein, a recombinant E. coli transformed by the plasmid, a preparation method of Mycobacterium tuberculosis RmlC recombinant protein using E. coli, a purification method of recombinant RmlC protein prepared by said method, and the RmlC protein purified by said method.

### BACKGROUND ART

Approximately 1.7 billion people, 32% of the world population, are infected by tuberculosis. There are 8 million new patients every year and about 34% of them, 2.7 million, died from this serious disease. In Korea, it is estimated that there are approximately 0.7 million tuberculosis patients, 0.14 million new patients every year, and 4,000 people died from the disease. Therefore, tuberculosis remains as a serious health problem.

Tuberculosis is a chronic infectious disease caused by Mycobacterium tuberculosis and currently the prevalence is increasing worldwide as a complication, of acquired immunodeficiency syndrome(AIDS). Also, many of the recent Mycobacterium tuberculosis has multiple-drug resistance that makes tuberculosis treatment more difficult.

Therefore, to eradicate tuberculosis, various efforts are needed such as development of a rapid diagnostic technique, development of a new drug, molecular epidemiological study and also study on vaccine development. Among those, development of a new drug is one of the most urgent for the purpose.

The main classical method for the antibiotics development was random screening of an antibacterial molecule(s) from microorganisms or natural materials. However, this approach has disadvantage such as much time and investment.

Recently, as a more advanced approach, new drug development is being performed. This approach includes the study of metabolic processes of corresponding microorganism and targeting an essential enzyme for its survival. It is desirable that the target enzyme is specific to the corresponding microorganism and does not exist in the cell of mammalians including human being. However, this approach has similar disadvantages with the previously described method in terms that it needs screening of materials that inhibit the target enzyme activity.

As a method to overcome these problems, Kuntz et al. proposed the concept of “structure-based drug design” in early 1990. This is a strategy to design and synthesize an inhibitor of a target enzyme by determining the structure of a target enzyme. The time required for the development of a new drug can be substantially shortened by this approach. This approach needs understanding of metabolic pathway that a target enzyme involves, cloning of the corresponding gene, overexpression, and purification in the first place. The purified enzyme can be used for crystal formation and structure determination by X-ray crystallography. Therefore, the 4-step procedure below is required to develop a new drug for tuberculosis based on the concept explained above.

First, it needs cloning of target enzyme gene, overexpression, purification, and establishment of activity detection method of the corresponding enzyme.

Second, it needs crystal formation, structural analysis of the purified target enzyme, and design and synthesis of enzyme inhibitors.

Third, it needs demonstration of inhibition effect of enzyme activity of the inhibitors designed and synthesized and selection of new drug candidates through evaluation of the bactericidal activity against Mycobacterium tuberculosis.

Fourth, it needs animal test and clinical test of new drug candidates selected.

The First thing for the development of new anti-tuberculosis drugs according this new concept is to screen components that are involved in the pathogenicity of or vital to Mycobacterium tuberculosis and the corresponding genes.

One of the factors that contribute to the pathogenicity of Mycobacterium tuberculosis is its thick cell wall. It contributes to antibiotics resistance and functions as a permeability barrier. So far, a lot of information has been accumulated about the cell wall structure of acid-fast bacteria where Mycobacterium tuberculosis belongs. The cell wall of Mycobacterium tuberculosis has a basic structure that includes two large polymers named peptidoglycan and arabinogalactan. These two polymers are linked by a covalent binding. Ethainbutol, currently being used as an anti-tuberculosis drug, is known to exhibit anti-tuberculosis activity by inhibition of arabinogalactan synthesis in Mycobacterium tuberculosis. Meanwhile, the base unit of wax structure, which contributes to the hydrophobicity of cell wall of Mycobacterium tuberculosis, is known to be mycolic acid. The mycolic acid is linked to arabinogalactan through covalent binding and arabinogalactan is again linked to peptidoglycan layer. According to a molecular structural study on the cell wall of Mycobacterium tuberculosis, there is a very small bridge-like structure between arabinogalactan and peptidoglycan. Thus these two polymers are connected to each other by this structure. In more detail, galactan part of the arabinogalactan and muramyl residue of peptidoglycan are linked by the bridge structure of [L-Rhamnose-N-Acetylglucosamine-Phosphate] (Reference: FIG. 1). Therefore, if the biosynthesis of either Rhamnose or N-acetylglucosamine that are the components of this bridging structure is blocked, cell wall of Mycobacterium tuberculosis gets weakened and, as a result, the material that has this effect could be a new drug candidate for tuberculosis treatment.

Among the components of this bridge structure Rhamnose is one of the components of O antigen, a component of lipopolysaccharide that is existed in cell wall of enteric bacteria. Numerous studies have been performed about the biosynthesis of Rhamnose in enteric bacteria. According to these studies, Rhamnose is synthesized as a form of deoxythymidine diphosphate(dTDP)-Rhamnose, and dTDP-Rhamnose is known to be the molecule providing Rhamnose to the cell wall of both Mycobacterium tuberculosis and general bacteria. These studies showed biosynthesis pathway of dTDP-Rhamnose in enteric bacteria, dTDP-Rhamnose is synthesized from deoxythymidine triphosphate(TTP) and glucose-1-phosphate, and 4 genes of rmlA, rmlB, rmlC and rmlD are involved. RmlA protein is glucose-1-phosphate thymidylyltransferase that produces dTDP-glucose from α-D-glucose-1-phosphate and dTTP. RmlB protein is dITDP-4,6-dehydratase that produces dTDP-4-keto-6-deoxyglucose from dTDP-glucose. RmlC protein is dTDP-6-deoxyglucose-3,5-epimerase that produces dTDP-4-keto-6-deoxymannose from dTDP-4-keto-6-deoxyglueose. RmlD protein is dTDP-6-deoxymannose dehydrogenase that produces dTDP-Rhamnose from dTDP-4-keto-6-deoxymannose. (Reference: FIG. 2)

In this respect, studies on genes and proteins that are involved in biosynthesis of dTDP-Rhamnose are very important as targets for the new drug development against tuberculosis. Currently, studies on new drug development targeting the dTDP-Rhamnose synthesis are in its very early stage in Mycobacterium tuberculosis. This is due to lack of information on metabolic pathways to synthesize dTDP-Rhamnose as well as information on enzyme and corresponding genes.

Meanwhile, it is ideal that only a single copy of the drug target gene or enzyme exists in the corresponding organism. According to the whole nucleotide sequence information of Mycobacterium tuberculosis standard isolate H37Rv that are revealed in 1998, rmlA and rmlB have more than two copies while rmlC and rmlD have only one copy. Therefore, it showed that the target genes for the new drug development through inhibition of dTDP-Rhamnose biosynthesis are mostly likely to be rmlC and rmlD. (Reference: Cole S. T. et al. Nature, 393:537-544(1998))

### DISCLOSURE OF THE INVENTION

As a basic step to develop a new drug against tuberculosis that inhibits dTDP-Rhamnose, a target molecule, the inventor made an effort to express rmlC in large quantities. As a result, the inventor confirmed that E. coli transformed with a recombinant plasmid that contains rmlC gene of standard Mycobacterium tuberculosis H37Rv produces RmlC protein of its natural state without the fusion of unnecessary amino acids as well as that corresponding RmlC protein contained its enzymatic activity.

After all, the first objective of this invention is to provide a recombinant plasmid that can produce RmlC protein of Mycobacterium tuberculosis in large quantities.

The second objective of this invention is to provide recombinant E. coli that is transformed by the said plasmid.

The third objective of this invention is to provide a preparation method of recombinant RmlC using the said E. coli.

The fourth objective of this invention is to provide purification method of the recombinant RmlC protein that is produced as described above.

The fifth objective of this invention is to provide purified RmlC protein using the said purification method of recombinant RmlC protein.

### BRIEF DESCRIPTION OF THE DRAWINGS

FIG. 1 is a supplementary picture describing the structure of cell wall of Mycobacterium tuberculosis.

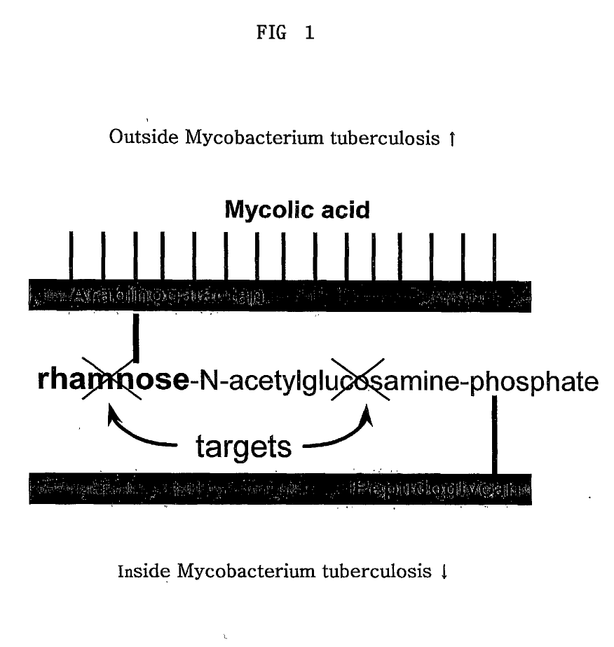

FIG. 2 describes the biosynthesis pathway of Rhamnose.

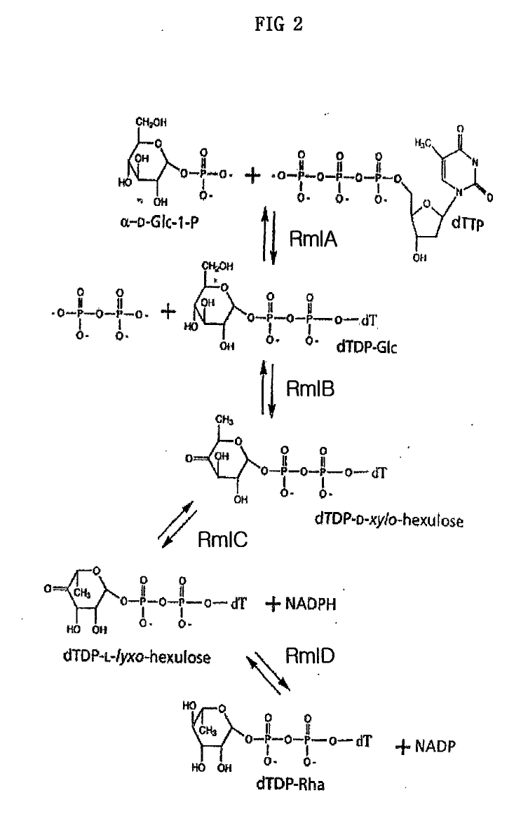

FIG. 3 is a picture of agarose gel electrophoresis presenting 609 bp rmlC gene that are amplified by PCR with genome DNA of standard Mycobacterium tuberculosis H37Rv as a template.

FIG. 4 is a picture of SDS-PAGE electrophoresis presenting purification of RmlC protein from E. coli that is transformed with recombinant plasmid pRMC609.

FIG. 5 is a chromatogram presenting the result of enzymatic activity measurement of the RmlC protein using High Performance Liquid Chromatography (HPLC)

### BEST MODE FOR CARRYING OUT THE INVENTION

The following is a more detailed explanation of the present invention.

In this invention, for the preparation of the recombinant protein RmlC, genomic DNA is prepared from H37Rv first. Then the rmlC gene is amplified by PCR using this genomic DNA as a template. A recombinant plasmid is constructed that expresses RmlC protein in large quantities by cloning the amplified rmlC gene on pET23b, a gene overexpression plasmid. An E. coli is transformed by this recombinant plasmid and the recombinant RmlC protein of Mycobacterium tuberculosis is prepared from the transformant in large quantities through the process of cultivation and induction.

Meanwhile, to separate RmlC protein expressed from the said transformant, the protein expression is induced by cultivation of the transformant and the supernatant of the transformant protein is fractionated by anion exchange resin chromatography. The fraction that contains RmlC protein is warmed between 50° C. and 80° C., ideally at 65° C., and the supernatant obtained is fractionated by FPLC gel-filtration chromatography.

Next, the size of the purified recombinant RmlC protein on an electrophoresis gel is confirmed by calculating the estimated protein size deduced from the nucleotide sequence of the rmlC gene. The enzymatic activity of the recombinant RmlC protein is examined by HPLC whether it retains its natural state. As a result, the RmlC protein prepared and purified by the said method in this invention does not have unnecessary amino acids and exhibits enzymatic activity of RmlC itself. The size of recombinant RmlC protein comprised of the same amino acid sequences as those of RmlC in its natural state is smaller than the previously reported recombinant RmlC protein that has 15 additional unnecessary amino acids. Therefore, it is clear that the efficiency of mass preparation and purification of smaller RmlC is greater than those of large RmlC protein when identical or similar mass preparation and purification methods are applied. Also, purified RmlC protein that are prepared and purified by this method is closer to Mycobacterium tuberculosis RmlC protein of its natural state. Thus, higher accuracy is expected when it is used for crystal formation and third dimensional structure determination by X-ray crystallography.

The following will be more detailed explanation of the present invention by examples.

These examples are only to explain the present invention and embodiments of the present invention are not limited only to the above, and it is evident that it can be diversely modified by a person who has ordinary knowledge in the appropriate field, within the technical idea of the present.

### EXAMPLE 1

### Culture of Mycobacterium tuberculosis

One loop amount of standard Mycobacterium tuberculosis isolate H37Rv, that has been cultured and stored in Ogawa solid medium, is inoculated to Middlebrook 7H9 liquid medium (Difco, Co. USA) with ADC (Albumin fraction V: bovine, Dextrose and Catalase: Difco, Co. USA). The culture is incubated for more than 4 weeks with light shaking (about 100 rpm) at 35-37° C. until enough bacteria can be obtained. Middlebrook 7H9 liquid medium is made by dissolving 4.7 g of medium powder in 900 mL of distilled water followed by autoclave. After autoclave, 2 mL of glycerol, that is autoclaved separately, and 10 mL of Middlebrook ADC is added.

The bacteria are obtained through centrifugation of the culture at 3000×g for 20 minutes. A portion of the bacteria is kept at −70° C. for storage after addition of Brucella broth and 15% glycerol. The rest of the bacteria portion is kept at −20° C. and is used for various experiments.

### EXAMPLE 2

### Genomic DNA Separation from Mycobacterium tuberculosis Cultured in Large Quantities

Mycobacterium tuberculosis cultured in large quantities from example 1 is collected and used for DNA preparation after incubating at 75° C. for 20 minutes for virulence attenuation. First, the bacteria are frozen at −70° C. and thawed. Then it was left at 37° C. for 1 hour in the presence of 2 mg/mL of lysozyme. Then it was left at 55° C. for 48 hours in the presence of 1% SDS and 1 mg/mL of proteinase K. The lysate is washed twice at 55° C. for 30 minutes with TE buffer solution that contains 0.04 mg/mL of Phenylmethylsulfonylfluoride, an inhibitor of proteinase K. Then, equal amount of chloroform-isoamylalcohol mixture (chloroform:isoamylalcohol=24:1(v/v)) is added, mixed well and centrifuged. The upper liquid portion is taken and the genomic DNA is precipitated by adding 0.6 times volume of isopropanol.

### Example 3

### Amplification of Mycobacterium tuberculosis rmlC Gene by PCR

Mycobacterium tuberculosis rmlC gene is amplified by PCR using synthesized primer set p1/p2 (p1: sequence number 1; p2: sequence number 2) with genome DNA of Mycobacterium tuberculosis prepared in example 2 as a template.

As seen in sequence number 1 and 2, there are EcoRI (GAATTC) and NdeI (CATATG) cut-off regions that contain initiation codon (ATG) at 5′-terminus of p1. There are BamHI cut-off region (GGATCC) and CTA sequence that is complementary base sequence to termination codon (TGA). EcoRI, NdeI, and BamHI cut-off regions in p1 and p2 are for the effective cloning of PCR product to the plasmid after PCR.

PCR reaction mixture contains 20 mM Tris-HCl (pH 8.8), 10 mM KCl, 2 mM (NH₄)₂SO₄, 0.1% Triton X-100, 0.5 μM of each primer, 200 μM of dNTPs, and 2.5 unit of Vent DNA Polymerase (New England Biolabs, Inc., USA). PCR is performed after 1 ng of template DNA is added to the reaction mixture with a total volume of 50 μl. To prevent water evaporation during the reaction, mineral oil was dropped on the reaction mixture. PCR temperature cycle contains once for 5 minutes at 94° C.; 30 seconds at 96° C., 30 seconds at 60° C., and 30 seconds at 72° C.(25 times); and once for 7 minutes at 72° C.

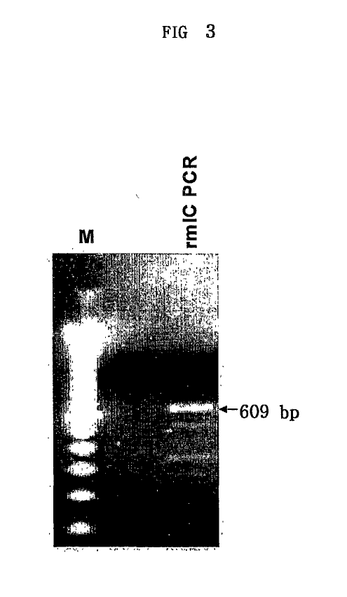

FIG. 3 is the picture of agarose gel electrophoresis showing the 609 bp rmlC gene amplified by PCR. In FIG. 3, M is 100 bp ladder DNA and is a standard DNA for estimating the size of DNA. As seen in FIG. 3, the expected 609 bp band was observed.

### Example 4

### Mass Expression and Purification of Mycobacterium tuberculosis RmlC protein

The rmlC gene amplified in Example 3 does not have errors during DNA synthesis, which can be occurred during PCR reaction. Amplified rmlC gene is digested with restriction enzyme NdeI and BamHI, and is cloned to pET23b (Novagen Co., USA), a mass expression vector, and the recombinant plasmid is named pRMC609.

Plasmid pRMC609 is introduced to BL21 (PlysS) E. coli, a host strain of expression vector pET23b. The E. coli BL21 (PlysS) that are transformed with pRMC609 is named BRMC609, and deposited to Korea Culture Center of Microorganisms (KCCM) on Jul. 3, 2000 (Deposit Number KCCM-10192).

BRMC609 transformant is cultured in 3 liters of LB liquid medium that contains 50 μg/mL of ampicillin and the expression of the protein is induced at 37° C. for 4 hours by adding 0.5 mM IPTG (Isopropylthiogalactoside) at late logarithmic phase.

Then E. coli cell is obtained by centrifuge and recombinant RmlC protein is prepared and purified using the chromatography method as follows. That is, to E. coli cells obtained, 1 mM EDTA, 0.1 mM PMSF, 1 μg/mL leupeptin, 0.1 mg/mL lysozyme, and 200 mL of 20 mM Tris-HCl buffer solution (pH 7.5) that contains 1 μg/ml DNase are added, and the bacteria is dissolved after repeated freezing and thawing. After centrifugation at 15,000 rpm for 20 minutes, the supernatant is placed in a DEAE Sepharose CL 6B column that is equilibrated by 20 mM Tris-HCl buffer solution (pH 7.5) containing 2 mM EDTA. The protein is eluted with a straight-line concentration gradient of NaCl of 0 to 0.5M that is dissolved in 10 mM Tris-HCl buffer solution (pH 7.5). The fractions that contain RmlC protein are checked by Coomassie blue staining after SDS-PAGE.

After collecting fractions that contains recombinant RmlC protein, the purity of the protein is increased using its heat stability. The RmlC protein solution is centrifuged at 12,000 rpm for 20 minutes after incubation in 65° C. water bath for 10 minutes, and the liquid is concentrated using Centriprep 10 concentrator (Amicon Co., USA)

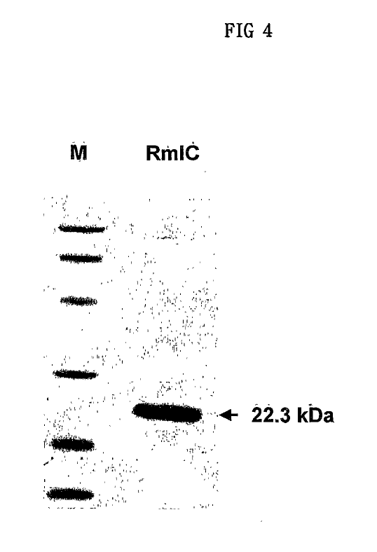

The final purification is FPLC gel-filtration chromatography. The Superdex column is equilibrated with 20 mM Tris-HCl buffer solution (pH 7.5) that contains 0.1 M NaCl and 0.02% sodium azide. The fractions that contain RmlC protein are examined by Coomassie blue staining after SDS-PAGE. After collecting fractions that contain recombinant RmlC protein, the protein is finally concentrated using Centriprep 10 concentrator (Amicon Co., USA). As a result, the 22.3 kDa protein is confirmed to be purified as estimated from the nucleotide sequence of rmlC gene. (Reference: FIG. 4)

### Example 5

### Measurement of Enzymatic Activity of RmlC Protein Using High Performance Liquid Chromatography

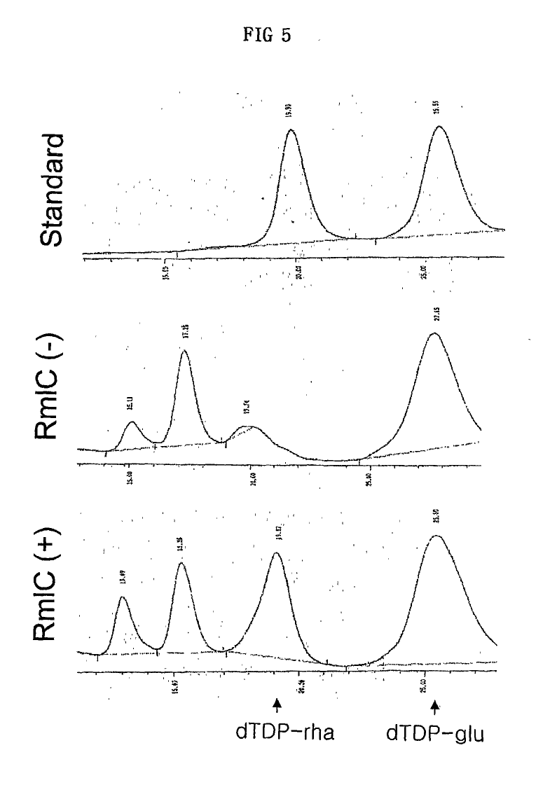

Enzymatic activity of the purified RmlC protein in example 4 is examined by High Performance Liquid Chromatography (HPLC). In the analyzing mixture, 2 mL of dTDP-Glucose, 6 nmol of NADPH, 50 μg of crude soluble protein obtained from E. coli BW24970 (RmlB and RmlD proteins that are involved in making dTDP-Rhamnose from dTDP glucose are contained here), 2 μg of purified RmlC protein and 1 nM, of MgCl₂ are dissolved in 50 mM Hepes buffer solution (pH 7.6). After this mixture solution is incubated for 1 hour at 37° C., 67 μL of ethanol is added. Denatured protein is removed by centrifugation at 14,000×g for 10 minutes, and the supernatant is placed in Dionex PA-100 HPLC column (Dionex Co., USA). Then the column is eluted by 75 mM KH₂PO₄ and absorbance at 254 nm is measured. Synthesis of dTDP-Rhamnose can be confirmed using dTDP-glucose and dTDP-Rhamnose as standards. The result shows the synthesis of dTDP-Rhamnose only in the reaction where purified RmlC protein was added not in the reaction where RmlC protein was not added. (Reference: FIG. 5)

As explained and confirmed above, according to the present invention, the RmlC protein expressed in large quantities from a recombinant plasmid (this contains Rhamnose biosynthesis gene rmlC, a known target for new drug development against Mycobacterium tuberculosis) is an RmlC protein of its natural state without the fusion of unnecessary amino acids and retains the enzymatic activity of corresponding protein. Therefore, recombinant plasmid, its transformant, preparation and purification method of the recombinant RmlC protein presented in this invention is very effective in mass preparation of Rhamnose biosynthesis enzyme RmlC, and the RmlC protein that is prepared in large quantities and purified using said method can be very useful in protein crystal formation and three dimensional structural determination of RmlC protein by X-ray crystallography that are necessary steps for structure-based new drug development.

1. Plasmid pRMC609 that contains Rhamnose biosynthesis gene rmlC of Mycobacterium tuberculosis on general expression vector pET23b

2. E. coli that is transformed with the plasmid pRMC609

3. A transformant (KCCM-10192) of claim 2, wherein the host strain is E. coli BL21 (PlysS)

4. A manufacturing method of recombinant RmlC protein comprising steps of:
culturing the transformant of claim 2; and
inducing expression of Rhamnose biosynthesis gene rmlC of Mycobacterium tuberculosis.

5. A purification method of recombinant RmlC protein comprising steps of:
(i) culturing the transformant of claim 2 and inducing expression of Rhamnose biosynthesis RmlC protein;
(ii) lysing the transformant the expression of which is induced in the step (i) and obtaining and anion-exchange chromatographing the supernatant;
(iii) collecting the fractions of the step (ii) that contain recombinant RmlC protein and obtaining the supernatant by warming the fraction between 50-80° C.; and
(iv) FPLC gel-filtration chromatographing the supernatant obtained in the step (iii).

6. Rhamnose biosynthesis RmlC protein of Mycobacterium tuberculosis purified by the purification method of RmlC protein of claim 5.


SEQUENCE LISTING (2 sequences)

SEQ ID NO: 1 - DNA, 34 bp, Artificial Sequence
  Single stranded oligonucleotide primer
ggaattccatatgaaagcacgcgaactcgacgtc

SEQ ID NO: 2 - DNA, 28 bp, Artificial Sequence
  Single stranded oligonucleotide primer
cgggatcctaggtgccgcgcatctcccc